(19) United States
(12) Patent Application Publication
(10) Pub. No.: US 2025/0104802 A1
(43) Pub. Date: Mar. 27, 2025

(54) DYNAMICALLY SIZED WINDOWS BASED ON THREE-DIMENSIONAL PROTEIN STRUCTURES

(71) Applicant: Helix, Inc., San Mateo, CA (US)

(72) Inventor: Elizabeth Cirulli Rogers, Lakeside, CA (US)

(21) Appl. No.: 18/372,527

(22) Filed: Sep. 25, 2023

(51) Int. Cl. G16B 15/20; G16B 20/20

(52) U.S. Cl. CPC G16B 15/20; G16B 20/20

(57) ABSTRACT

Various embodiments disclosed relate to a method of facilitating analysis of variants via consideration of three-dimensional spatial positions on a protein coded by a gene. A method may include receiving a three-dimensional protein structure of the protein encoded by the gene. A method may include selecting a candidate spatial position at the three-dimensional protein structure. A method may include identifying variants within the gene. A method may include mapping locations of the variants along a sequence of the gene to variant spatial positions on the three-dimensional protein structure. A method may include calculating three-dimensional distances between the candidate spatial position and each of the variant spatial positions on the three-dimensional protein structure. A method may include selecting a subset of the variants for inclusion within a statistical window, based on the three-dimensional distances for corresponding variant spatial positions.

### TECHNICAL FIELD

The subject matter disclosed herein generally relates to collection and processing of genetic material. More specifically, the subject matter herein relates to identifying and reporting the variants of a population of individuals that are associated with a specific trait.

### BACKGROUND

Genetic material can be collected and processed for various forms of bioinformatic analysis. Once processed, genetic information gleaned from genetic material can be used in a variety of bioinformatic analyses. For example, genetic information can be analyzed to identify and report variants in a population, such as genetic variants associated with specific traits or phenotypes.

### SUMMARY OF THE DISCLOSURE

In some aspects, the techniques described herein relate to a method for facilitating analysis of variants via consideration of three-dimensional spatial positions on a protein coded by a gene, the method including: receiving a three-dimensional protein structure of the protein encoded by the gene; selecting a candidate spatial position at the three-dimensional protein structure; identifying qualifying variants within the gene; mapping locations of the qualifying variants along a sequence of the gene to variant spatial positions on the three-dimensional protein structure; calculating three-dimensional distances between the candidate spatial position and each of the variant spatial positions on the three-dimensional protein structure; and selecting a subset of the qualifying variants for inclusion within a statistical window, based on the three-dimensional distances for corresponding variant spatial positions.

In some aspects, the techniques described herein relate to a non-transitory computer readable medium containing program instructions for causing a computer to perform the method of: receive a three-dimensional protein structure of the protein encoded by the gene; select a candidate spatial position at the three-dimensional protein structure; identify qualifying variants within the gene; map locations of the qualifying variants along a sequence of the gene to variant spatial positions on the three-dimensional protein structure; calculate three-dimensional distances between the candidate spatial position and each of the variant spatial positions on the three-dimensional protein structure; and select a subset of the variants for inclusion within a statistical window, based on the three-dimensional distances for corresponding variant spatial positions.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts a diagram of an example bioinformatics analysis system.

FIG. 1B is a flow chart illustrating an example method of using a bioinformatics analysis system.

FIG. 2A is a flow chart illustrating a method of intaking and preparing a genetic sample in a wet laboratory for bioinformatic analysis in an example.

FIG. 2B is a flow chart illustrating a method of intaking a genetic sample in a wet laboratory for bioinformatic analysis in an example.

FIG. 2C is a flow chart illustrating a method of preparing a genetic sample in a wet laboratory for bioinformatic analysis in an example.

FIG. 3A illustrates a flow chart depicting a method of facilitating analysis of variants via consideration of three-dimensional spatial positions on a protein coded by a gene in an example.

FIG. 3B illustrates a flow chart of a method of selection candidate spatial positions in an example.

FIG. 4A illustrates a schematic diagram of a genetic sequence and corresponding amino acid sequence in an example.

FIG. 4B illustrates a schematic diagram of a protein folding in an example.

FIG. 5 illustrates a flow chart of a method of iteratively facilitating analysis of variants via consideration of three-dimensional spatial positions on a protein coded by a gene in an example.

FIG. 6 illustrates a schematic diagram of window selection in an example.

FIG. 7 illustrates a flow chart of selecting qualifying variants by spatial proximity in an example.

FIG. 8 illustrates a schematic diagram of a computer in an example.

### DETAILED DESCRIPTION

The present disclosure describes, among other things, methods of using a statistical sliding window (e.g., a “power window”) for analyzing a three dimensional space of a protein encoded for by a gene. The method identifies 3D spatial positions in a 3D structure of a protein, and groups genetic variants for analysis based on the 3D spatial positions.

For each of the 3D spatial positions, qualifying variants are added to the statistical window. The variants that code for amino acids physically closest to the 3D spatial position are added to the window first until a threshold number of variants (expressed by a corresponding number individuals), based on a desired statistical power, is added to the window.

As analysis continues, the spatial size of the window relative the structure of the protein varies throughout analysis depending on the number and location of nearby amino acids changed by qualifying variants. Once a window has reached that desired number of qualifying variants, analysis can be run. In some cases, several windows are built, and then analysis is run.

This can help capture interactions between regions of the gene that code for amino acids that are physically close in the protein, even if the regions are located far apart from each other according to the linear sequence of the gene. The captured interactions can help reveal important information about genetic variants and their impacts on patient health.

A variety of techniques have been used for identifying genetic variants having clinical implications. Genome-Wide Association Study (GWAS) is such a technique used to identify associations between genetic variants and traits (e.g., phenotypes). GWAS can start with acquiring genomic data and population trait data. Then, genetic variants within the population can be identified. These genetic variants can be associated with the traits of interest. Genetic variants that appear with a different (e.g., greater or lesser) allele frequency within the portion of the population expressing that trait compared to the entire population are expected to be associated (e.g., positively or negatively) with that trait. But GWAS has limitations in precision.

Where limitations are found with regards to variants that are rare, gene-based collapsing analysis can be used instead of GWAS. Gene-based collapsing analysis can analyze a small portion of the genome at a time for statistical analysis instead of analyzing each genetic variant individually. However, selecting the exact portion of the genome to be considered for statistical purposes can be challenging. For example, this approach can be limiting when attempting to determine the influence of a rare variant on a trait; it remains difficult to determine exactly which rare variants to group together for analysis.

“Sliding window” analysis techniques (for example, the recently developed “power window” method) can help identify associations between genes and traits (e.g., phenotypes) by dynamically controlling the size along a portion of a chromosome of a window of variants considered for statistical analysis. Such sliding window approaches dynamically vary the window size (rather than the number of qualifying variants considered) during analysis to help the analysis be performed with a requisite amount of statistical power. For example, the size of the window is scaled to maintain a consistent range of individuals carrying qualifying variants as the window advances through genomic coordinates. For example, the size of the window can be scaled according to a number of qualifying variants, bases, exons, or another indicator. The number of variants encompassed by the window can be dynamically adjusted as analysis is performed. However, additional methods for greater insights between genetic variant and clinical outcomes are desired.

Discussed herein, the power window concept can be applied to the three-dimensional space of a protein encoded for by a gene. The methods discussed herein can allow for obtaining the three-dimensional structure of a protein encoded by a reference version of a gene and identifying three-dimensional spatial positions of interest.

For each identified three-dimensional spatial position, associated qualifying variants can be identified and added to the statistical analysis window. Qualifying variants can be added to the window starting with those which code for amino acids physically closest to the three-dimensional spatial position. Qualifying variants can be added to the window until a number of qualifying variants reaching a predefined statistical power are included.

Thus, the spatial size of the window relative to the three-dimensional structure of the protein varies throughout analysis depending on the number and location of nearby amino acids coded by qualifying variants. Once the window has reached the desired number of qualifying variants, analysis can be run on those qualifying variants. The analysis can help to capture interactions between regions on the gene that code for amino acids that are physically close in the protein even if those regions are located far apart from each other according to the linear sequence of the gene.

Differences in protein structure resulting from genetic variants can have notable and clinically relevant impact. Analyzing portions of a gene that code for three-dimensionally physically proximate regions of the protein can allow for better understanding of the potential impact of these genetic variants.

### Definitions

As used herein, “accession”, or “accessioning” refers to receiving and preparing a sample for later laboratory processes.

As used herein, “amplifying” refers to the production of multiple copies of a sequence of nucleic acid or other genetic material, such as RNA or DNA.

As used herein, “bioinformatics” refers to the science of collecting complex biological data such as genetic codes.

As used herein, “biological sample”, or “sample” refers to a specimen from a patient, such as for bioinformatic research.

As used herein, “contamination” refers to a sample that is impure, polluted, or unsuitable for biological analysis and research.

As used herein, “genetic material” refers to a fragment, molecule, or a group of nucleic acids, such as DNA or RNA, genetic material from one or more chromosomes, mitochondrial genetic material, or other genetic material.

As used herein, a “locus” refers to a single position on a chromosome or fragment of genetic material (e.g., as indicated by a genomic coordinate).

As used herein, “mutation” refers to a changed structure of a gene that results in a variant form of the gene (e.g., with respect to a reference genome).

As used herein, “pathogen” refers to a bacterium, virus, or other microorganism that can cause disease.

As used herein, “read” or “read pair” refers to data that defines a DNA or RNA sequence from a fragment or section of genetic material.

As used herein, “sequencing” refers to a process of determining the nucleic acid sequence, an/or the order of nucleotides in genetic material.

As used herein, “variant” or “genetic variant” refers to a subtype that is genetically distinct from other subtypes.

### Bioinformatic Analysis System and Methods

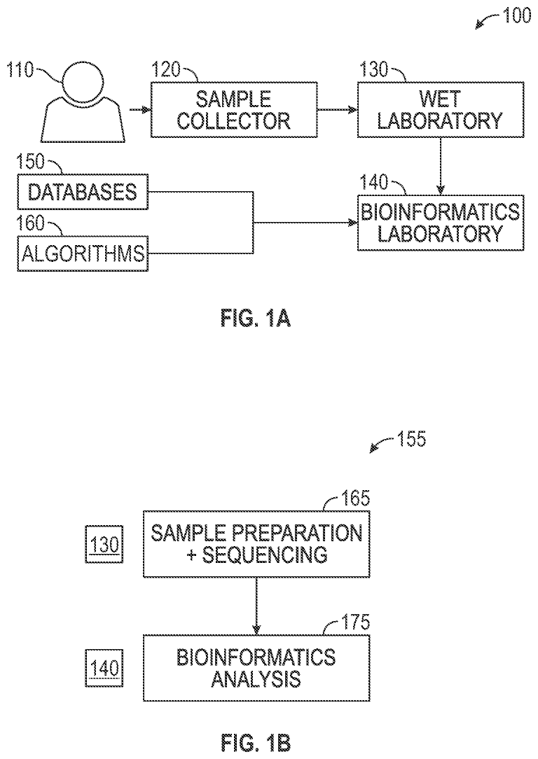

FIG. 1A depicts a diagram of an example bioinformatics analysis system 100, while FIG. 1B is a flow chart illustrating a method 155 of using such a bioinformatics analysis system 100.

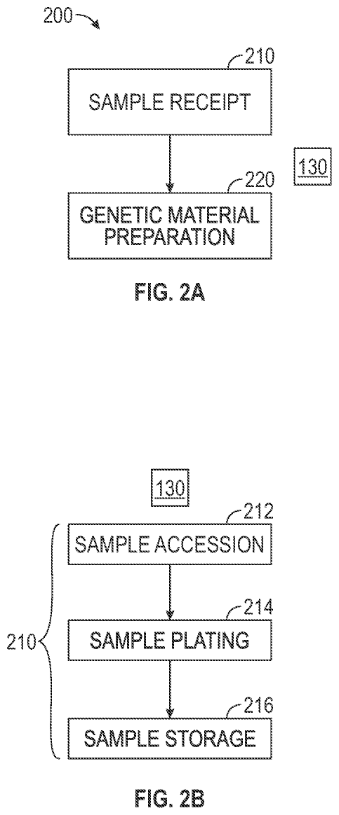

The system 100 can include both physical or “wet” laboratory components, and bioinformatics components. For example, the system 100 can interact with patients 110, from whom biological samples can be collected, in addition to sample collectors 120, which may be, for example, doctors' offices, pharmacies, or other appropriate places where patient samples can be taken. The system 100 includes a wet laboratory 130 which is positioned to receive the biological samples and process those samples to produce sequenced genetic material for analysis, such as at step 165 of method 155. These methods of sample receipt, handling (e.g., accessioning), and sequencing, are discussed in detail below with reference to FIGS. 2A to 2C.

The system 100 can additionally include data driven components, such as databases 150 and algorithms 160 or other programs that support a bioinformatic laboratory 140 used to analyze genetic information. These data driven components can be used to do bioinformatic analysis (step 175 in method 155). Specific examples of such bioinformatic analysis are discussed in detail below.

### Sample Processing Methodology

Before bioinformatic analysis, biological samples are collected and sequenced through physical components of the system 100, such as through the wet laboratory 130. Methods of receiving and processing such samples are summarized in FIGS. 2A to 2C. FIG. 2A is a flow chart illustrating a method 200 of intaking and preparing a sample for sequencing in a wet laboratory for bioinformatic analysis. The method 200 can include two primary portions: receiving the samples (step 210) and preparing genetic material from the samples (step 220). FIG. 2B illustrates portions of step 210, including a method of intaking a sample for sequencing in a wet laboratory for bioinformatic analysis. FIG. 2C illustrates portions of step 220 of the method 200, a method of preparing a sample in a wet laboratory for bioinformatic analysis.

The method 200 can begin with sample collection. For example, the samples can be collected by receiving a nasal swab, blood, saliva, or other material potentially containing genetic material of interest.

Accessioning Samples. Once received at the laboratory, at step 212, the samples can be accessioned, that is, prepared for later laboratory processes. For example, accessioning can include receiving a batch of samples. A batch of samples can include, for example, hundreds of individual samples, or thousands of individual samples. Each sample can be retained in a sample container. For example, test tubes can be used to store each of the samples. The sample containers can be sealed to help prevent environmental exposure and prevent sample co-mingling. For example, the sample containers may be sealed via a cap that is threaded, glued, press-fit, or otherwise affixed via appropriate sealing mechanism. When the samples are received in a batch, the corresponding sample containers may also include one or more remnants of a sampling tool, such as a swab used to collect the sample.

In some cases, the sample containers may be accompanied by Customer Sample Identifiers (CSI) such as by a component affixed to or integrated with the sample container. Such a CSI can uniquely distinguish individual sample containers from other sample containers being received. For example, a CSI may uniquely distinguish a sample from other samples in the same batch, other samples received on the same date, or other samples received from the same customer. Such CSI can be provided as a label such as a bar code or a Quick Response (QR) code, a chip such as a Radio Frequency Identifier (RFID), or another type of visual, transmission-generating, or other component affixed to or integrated with the sample container.

In some cases, the sample containers can be further sealed in an external container, such as a bag. External containers can help prevent contamination of samples, such as by preventing biological material from the samples contacting other or external surfaces. An external container can also help prevent cross-contamination between samples. Moreover, when a sample includes blood or a pathogen, the external container can provide an additional barrier to protect technicians who may handle the samples. The external container can additionally include documentation correlating to the CSI, such as information on the patient that the sample was sourced from, information indicating circumstances of sampling, for example, a sampling date, a sampling method, a location that the sample was acquired, a name or title for a person who performed the sampling, other information, or combinations thereof.

In some cases, the samples can be in a chemical solution. For example, the sample may be prepared in an aqueous solution, such as a saline solution. In some cases, the samples can include a bodily fluid such as saliva, mucus, blood, or other. In an example, the sample can have a volume of about 2 mL, of about 3 mL, of about 4 mL, or of about 5 mL.

The samples include genetic material. For example, the samples can include Deoxyribonucleic Acid (DNA) or Ribonucleic Acid (RNA). In an example, the genetic material is one or more of many constituent components within the sample. For example, one portion of the genetic material may exist within the nuclei or mitochondria of white blood cells that are included within the sample. In another example, another portion of the genetic material may exist within viruses or bacteria within the sample. In these types of examples, the genetic material is not yet isolated from the remaining constituent components of the sample. Thus, the genetic material should be isolated.

To begin isolating the genetic material, batches of the samples can be heated in ovens to facilitate cell lysis. The temperature and duration of heating can be chosen such that any pathogenic material within the samples is rendered harmless, such that cellular lysis occurs, or both. For example, the samples can be heated at a temperature of between about 40° C. and 80° C., or at a temperature of between about 15° C. and 200° C., or at another appropriate temperature range. The samples can be heated for a time period of about 30 minutes, or for a time period of about 50 minutes, or for another appropriate time period. In some cases, such as where the samples are the contents of a blood draw, the heating step may be skipped.

After heating, the batches of samples can be removed from the ovens. In an example, sample containers can be removed from external containers, such as by cutting open the external containers. The sample containers can be inspected, either in a manual, automated, or semi-automated fashion. For example, a technician or an automated system can determine the CSI for the sample and compare the CSI to documentation accompanying the batch. If there is a discrepancy between the CSIs on the sample container and in the documentation, the sample may be flagged as having an error condition. Similarly, if the CSI on the sample container is damaged (such as by abrasion, heat-damage, or water-damage) and has become unreadable, the sample may be flagged as having an error condition.

In some cases, the technician or automated system can further inspect the contents of the sample container, such as visually. If the sample does not include expected constituent components, then the sample can be flagged as having an error condition. For example, if the sample includes a fluid that is not permitted (such as extraneous blood), includes an entire swab or no swab, is within a fractured or broken sample container, or is outside of an expected range of volume (e.g., between two and five milliliters), or other conditions, then the sample can be flagged as having an error condition.

Subsequently, samples that have not been flagged with an error condition can proceed to sample integration. Here, the sample can be assigned a Laboratory Sample Identifier (LSI). Such an LSI can uniquely identify the sample from other samples received in the same batch, received on the same day, processed in the same laboratory, handled by the same company for sequencing, or combinations thereof. The LSI can be stored in a laboratory sample database, and uniquely correlated to the CSI for the sample. The LSI can be associated with any error codes reported from the sample. Both the CSI and the LSI can both be applied to the sample container.

Sample Plating. Once accessioned, the samples can be plated at step 214. At this point, the samples have been successfully integrated into the laboratory environment and are ready for analytics. The samples can next be prepared for transfer to a sample microplate. The sample microplate can be labeled with a unique identifier, which can distinguish the sample microplate from other sample microplates. For example, the sample microplate can be a solid body with about 50 wells to about 400 wells, distributed across rows and columns, each well having a capacity of about 30 μL to about 300 μL. In other examples, different size microplates with a different number of wells at varying volumes can be used.

The samples to be used on the microplate may be racked and the rack may be assigned an identifier, such as to allow a technician to understand which samples correspond to which LSIs. The technician may unseal the sample, such as by a manual, automated, or semi- automated tool to efficiently open the sample container. The tooling may, for example, unscrew, cut, or drill each sample container, to make the sample within available for physical transfer to the sample microplate.

The samples can then be transferred to the microplate, such as by an automated robot that operates an end effector in accordance with one or more programs for effective transfer of the samples. This can be done, for example, with a combination of actuators, piezoelectric elements, pressure systems, and/or other components operating the end effector of the robot. The end effector can uptake portions of the samples in micropipettes and transfer those samples to the corresponding wells in the microplate. In some cases, disposable tips can be used. In some cases, portions of the samples can be transferred. In some cases, reagents can be added to the samples. In some cases, controls can be included in the microplate. The sample microplate, once completed, can be transferred for further processing in the laboratory.

Sample Storage. After plating, the samples can be stored at step 216. In some cases, accessioned samples, plated samples, or other samples, are stored for later use. In this case, they can be stored at room temperature, or can be cryogenically frozen and arranged on racks for later retrieval. Samples can be preserved for periods of days or years to allow later rapid re-testing.

Extraction of Genetic Material. When genetic analysis is desired, the genetic material of the samples can be extracted for sequencing at step 222. In some examples, a reagent can be applied to sample wells to lyse cells therein to expose genetic material.

Additionally, aspirating, and dispensing reagents can be used to selectively bind genetic material released from lysed cells. In some examples, this can include applying a bead to the well. In this case, the beads can, for example, be magnetic beads that selectively bind to the genetic material. This can help allow for isolation and purification of the genetic material at the bead, leaving contaminants in the solution. In an example, a magnetic bead can be magnetically drawn to a magnetic base at or under the sample microplate. In this case, after the genetic material has been drawn to the bead, a flushing step can be performed to wash away remaining fluid, helping to remove impurities.

In some examples, fluid can be added or removed from wells, such as to concentrate or elute the genetic material. Fluid can be transferred from the wells of the sample microplate to a genome stock microplate. In an example, a portion of fluid can be removed from each well for quality control purposes. This can, for example, be used to determine concentration of genetic material therein.

Library Preparation. After extraction of the genetic material, a library can be prepared using the contents of the genome stock microplate at step 224. For example, the bead for each well, including ionically bonded genetic material, can be transferred to a distinct well of a library preparation microplate. The library preparation microplate can include an identifier. The LSI associated with each well on the sample microplate can be mapped to a corresponding well on the library preparation microplate. The library preparation microplate may be transferred to a new portion of the laboratory to help prevent amplified genetic material from entering portions of the laboratory where genetic material has not been amplified, which could result in contamination.

A reagent can be applied to each well of the library preparation microplate. The reagent can ionically bond to the surface of the bead within the well more strongly than the genetic material. This helps release the genetic material from the surface of the bead of each well, enabling the genetic material to be chemically interacted with.

Library preparation can include normalization of a concentration of genetic material in each well of the sample microplate. Library preparation can further include fragmentation of the genetic material via an enzyme or via the application of physical forces. During this process, the entire genome (e.g., roughly three billion base pairs for a human genome), may be fragmented into pieces. In an example, the pieces can be about 300 to 400 base pairs in length. These pieces can be referred to as nucleic acid fragments. These nucleic acid fragments can undergo adaptor ligation and indexing. In an example, this can include Next Generation Sequencing (NGS) library preparation processes.

The genetic material can then be amplified, such as by Polymerase Chain Reaction (PCR) amplification. The resulting solution can be purified and eluted. During this library preparation, one or more reference samples of genetic material can be added to the wells of the library preparation microplate. The reference samples can serve as controls and aid in quality control.

Once the library preparation has been completed, thousands or millions of distinct fragments of the genetic material, each corresponding with a different portion of a genome of the subject, can be ligated to predefined adapters that bind with the genetic material. Each of the adaptor ligated fragments is referred to as a “library.”

In additional examples, probes applied to each well can include chemical identifiers (“barcodes”) that are distinct from each other. The use of a different chemical identifier for probes applied to each well of the well plate can enable sequencing to later be performed for multiple subjects on the same flow cell, without conflating sequencing results for those subjects.

In additional examples, the library preparation process can further include controlling a concentration of the genetic material in each well, and purification and/or elution of the resulting material. Similar to the processes performed after extraction of genetic material, concentration of genetic material after library preparation can be confirmed for each well via testing.

Enrichment of Genetic Material. After library preparation, enrichment processes can be performed in order to either directly amplify (e.g., via amplicon or multiplexed PCR) or capture (e.g., via hybrid capture) predefined libraries of genetic material, such as at step 226 in FIG. 2C. This can enhance the ease of sequencing desired portions of the genome.

In an example, during enrichment, customized biotinylated oligonucleotide probes can be applied to the libraries. The probes can selectively hybridize genetic material occupying desired portions of the genome for the genetic material, such as specific genes, or the entire exome. Magnetic beads can bind to biotin molecules in the probes to attach the hybridized material to the magnetic beads. Magnetic forces can capture the beads in place, enabling remaining fluid within each well to be removed or washed out, thereby removing impurities, and leaving only the genetic material that is desired. Thus, genetic material can be released from the beads in a similar manner to that discussed above for prior processes.

In an example, hybrid capture target enrichment can be performed. During this process, the probes can include tailored oligonucleotides that are chosen to bind to the genetic material. The range of probes can be tailored as a group to bind to specific alleles, specific genes, the exome, the entire genome, etc. That is, each probe can bind to a nucleic acid fragment at a specific location on the genome, and the range of probes can be selected to ensure that alleles, genes, the exome, or the entire genome of the subject being considered is acquired.

In examples where probes are targeted to a portion of the entire genome, efficiency of the sequencing process is enhanced, by foregoing the need to sequence all of the roughly three billion base pairs found in the human genome.

The enrichment process can further include controlling a concentration of the genetic material in each well, and purification and/or elution of the resulting material. Similar to the processes performed after extraction of genetic material, concentration of genetic material after enrichment can be confirmed for each well via testing.

Sequencing of Genetic Material. After enrichment, the genetic material can be sequenced at step 228. Sequencing can be performed according to any of a variety of techniques, including short-read and long-read techniques.

In an example, the sequencing can be performed as Sequencing by Synthesis (SBS) at genetic analyzer equipment. For example, sets of enriched libraries of genetic material bound to probes in earlier steps can be transferred to a flow cell, and annealed to oligonucleotide probes within the flow cell. At this stage, the contents of multiple wells can be applied to the same flow cell, because the libraries within those wells are tagged with the chemical identifiers referred to above.

In an example, the chemical identifiers can include nucleotide sequences that are detectable during the sequencing process to determine a corresponding LSI. Complementary sequences can then be created via enzymatic extension to create a double-stranded portion of genetic material. The double-stranded genetic material can then be denatured, and the portion of the genetic material consisting of the library fragment can be washed away. Bridge amplification can then be performed to create copies of the remaining molecule in a localized cluster. For example, a cluster can comprise twenty to fifty copies of the same molecule, localized to a location the size smaller than a pinhead on the flow cell. Sequencing primers can be annealed to library adapters to prepare the flow cell for SBS. During SBS, the sequencing primer uses reverse terminator fluorescent oligonucleotides, one base per cycle, for several cycles in the forward direction. After the addition of each nucleotide, clusters can be excited by a light source, resulting in fluorescence which can be measured. The emission wavelength and signal intensity for each cluster determines a base call for that cluster. A chemical group blocking a 3′ end of the fragment can then be removed, enabling a subsequent nucleotide to be read. This can help control nucleotide addition and detection. After each cycle, denaturing and annealing can be performed to extend the index primer. A complementary reverse strand can be created and extended via bridge amplification. The reverse strand can then be read in the reverse direction for a number of cycles, in a manner similar to reads in the forward direction.

Throughout the processes discussed above, the laboratory environment can be carefully controlled to ensure quality. For example, temperature within each segment of the laboratory can be carefully monitored and controlled, and ultraviolet lighting or other features capable of inactivating genetic material can be carefully positioned to ensure that contamination does not occur.

In general, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text based FASTQ format, that reports quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics such as quality indicator metrics. The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Analysis of Variants With Three-Dimensional Spatial Positions

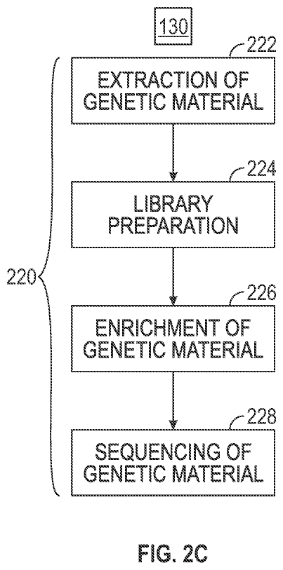

FIG. 3A illustrates a flow chart depicting a method 300 of facilitating analysis of variants via consideration of three-dimensional spatial positions on a protein coded by a gene in an example. The method 300 can include blocks 310 to 360.

The method 300 can begin by loading data for a gene of interest. At block 310, the method can include receiving a three-dimensional protein structure of the protein encoded by the gene. In some cases, the three-dimensional, folded structure of the protein itself can be received. For example, the three-dimensional structure of the protein can be predefined, such as by a research publication, a protein data bank, or other appropriate program. For example, Protein Data Bank (PDB) or AlphaFold databases may be used. In some cases, a program such as FoldX v5 can be used.

In some cases, data received can include a reference sequence for a gene. In this case, the method 300 can begin with receiving a reference sequence and producing such a corresponding three-dimensional protein structure, such as with a modeling tool to produce the structure.

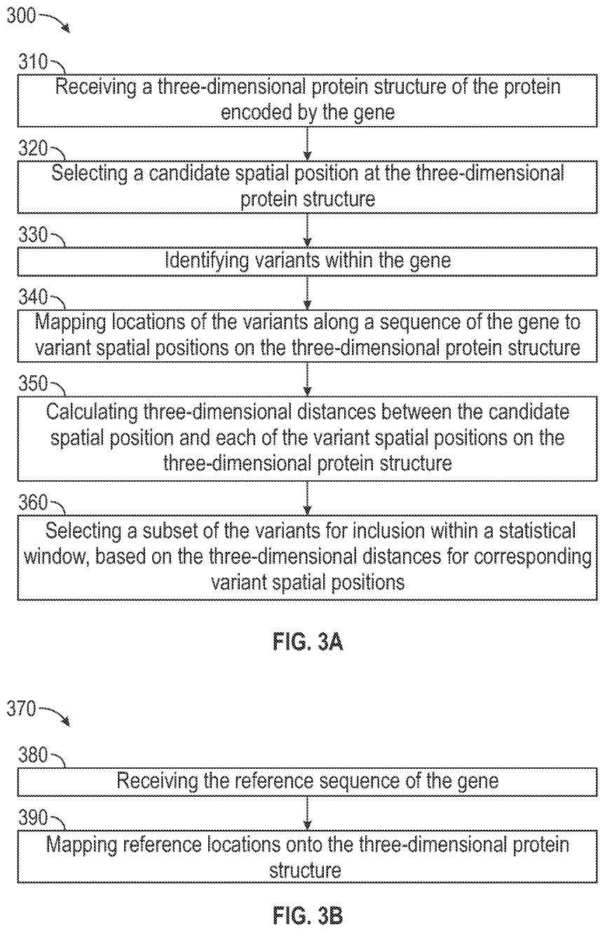

FIG. 4A illustrates a schematic diagram of a genetic sequence and corresponding amino acid sequence, while FIG. 4B illustrates a schematic diagram of a protein folding based on that genetic sequence. Here, FIG. 4A depicts a genetic sequence with a codon (ATG) corresponding to an amino acid methionine (MET). Meanwhile, in FIG. 4B, in the folded protein, the spatial position is very close to another nearby amino acid, which is not necessarily close to the corresponding portion of the genetic sequence.

In some cases, receiving the three-dimensional protein structure comprises receiving a partially defined three-dimensional protein structure, or a protein structure that is partially known, or a protein structure that changes or is changeable. For example, regions within a gene that are likely to be spatially adjacent to each other can be inferred via binding studies that identify parts of a protein that interact with each other or that interact with part of another protein. For example, a binding study may detect the presence of codons for a receptor of the protein in a first region, and may detect codons for a corresponding ligand in a second region that is separated from the first region along the linear sequence of the gene. In such a case, the first region and second region are known to code for spatially adjacent amino acids, even without the need for modeling the full 3D structure of the protein. Spatially adjacent regions may be included within a window for analysis, even without a fully realized determination of 3D protein structure, because they are known to be structurally coupled.

Spatial positions on the three-dimensional protein structure (e.g., in three-dimensional space) can be mapped to locations along the gene sequence. For example, specific loci on a gene sequence may define a codon for an amino acid within the protein. This amino acid may be associated with a specific three-dimensional coordinate on the structure of the protein. Such mapping can be done, for example, through the use of lookup data.

At block 320, the method can include selecting a candidate spatial position at the three-dimensional protein structure. The candidate spatial positions can be three-dimensional positions on the protein structure that are of interest; for example, the positions may be where a variant can be known to cause a change in trait or phenotype of interest. The candidate spatial positions can be selected in a variety of ways.

In some cases, the candidate spatial positions can be spatial positions along the protein structure that are already indicated in published literature as having an impact on health. In some cases, the candidate spatial position can be every spatial position along the protein that is associated with an amino acid. In some cases, the candidate spatial positions can include those corresponding to specific amino acids.

In some cases, the candidate spatial positions can include those that impact protein function. In some cases, the candidate spatial positions can include those that are identified by a functional screen.

In some cases, the candidate spatial positions are located with regard to an external molecule that interacts with the protein. For example, a candidate spatial position may be identified at a location where a protein binds to an external molecule such as cholesterol.

In some cases, the candidate spatial positions can be spatial positions of a gate surrounded by amino acids. In some cases, the candidate spatial positions can be spatial positions of a chamber surrounded by amino acids.

In some cases, the candidate spatial positions can be spatial positions located with regard to an external molecule that interacts with the protein. In some cases, the candidate spatial positions can be spatial positions at one or more interaction sites between regions of two or more proteins. In such an example, each candidate spatial position may be defined within a three-dimensional volume encompassing the various proteins being considered.

A single candidate spatial position can be selected, or multiple candidate spatial positions can be selected.

In some cases, these candidate spatial positions can be determined by receiving spatial information indicative of such a candidate spatial position, such as from literature. In some cases, these candidate spatial positions can be determined by mapping spatial positions that have been mapped from the gene sequence, such as mapping of a gene sequence to a three- dimensional protein structure briefly discussed above. FIG. 3B illustrates a flow chart of a method 370 of mapping and selecting candidate spatial positions in an example.

The method 370 can start at block 380, where the reference sequence of the gene is received. Then, at block 390 a series of reference locations from the reference sequence can be mapped on to the three-dimensional protein structure. Each reference location can be mapped to a different corresponding spatial position. The candidate spatial positions can be selected from these mapped positions.

This mapping can occur, for example, by unfolding the obtained three- dimensional protein structure and correlating the reference sequence of the gene to the three-dimensional protein structure, refolding the protein, and determining three-dimensional coordinates of the candidate spatial position.

Once the candidate spatial positions are selected, and their spatial positions confirmed, qualifying variants can be identified for the statistical window.

At block 330, the method can include identifying variants within the gene. The qualifying variants can be selected based on one or more selection criteria.

In some cases, identifying variants can include receiving population genomics data indicating locations of genetic variants. For example, such population genomics data can indicate, on a person-by-person basis, the nature and location of genetic variants carried by a person. Such population genomics data may include sequencing data. In some cases, the impact of these variants on health of a person may be unknown, such as particularly for persons with rare variants. Such population genomics data can indicate traits or phenotypes on a person-by-person basis.

For each of the selected candidate spatial positions, qualifying variants (expressed by corresponding individuals) can be selected within the population genomics data. As discussed above, a qualifying variant is a variant selected for study in order to determine whether a correlation exists between that variant and the trait being considered. The selected qualifying variants can be variants which meet selection criteria determined by a literature study, and may be rare variants. Variant filter criteria can be used to identify qualifying variants within the sequence data. For example, a qualifying variant can be a variant that alters the structure of a protein, such as a protein generated by the portion of the chromosome.

Qualifying variants can be stored along with identifying information for those qualifying variants. Such information may include a number of persons in the population carrying the qualifying variant, a list reciting individuals carrying the qualifying variant, a location of each qualifying variant within a portion of a chromosome, a sequence of each qualifying variant, etc.

The qualifying variants can include variants that meet criteria for analysis and are also within the portion of the chromosome selected for analysis. Identifying qualifying variants may include reviewing the sequence data of each individual, based on variant filter criteria. For example, variant filter criteria may define qualifying variants as variants that are coding (e.g., having base pairs that indicate stop_lost, mis-sense_variant, start_lost, splice_donor_variant, inframe_deletion, frameshift_variant, splice_acceptor_variant, stop_gained, or inframe_insertion) and also are not Polyphen benign or Sorting Intolerant From Tolerant (SIFT) benign. In such an embodiment, Polyphen benign may be considered any value less than 0.15, while SIFT benign may be considered any value that is greater than 0.05. In a further example, similar sequence pathogenicity algorithms may be utilized that are field-standard and used for such a purpose.

In another example, variant filter criteria may define qualifying variants as Loss of Function (LoF) variants (e.g., having base pairs that indicate stop_lost, start_lost, splice_donor_variant, frameshift_variant, splice_acceptor_variant, or stop_gained) or variants having other predicted molecular properties, such as mis-sense (a change in corresponding amino acid), splice site variants, etc. In such an embodiment, a variant may be required to be below a MAF cutoff of 0.1% in all Genome Aggregation Database (gnomAD) populations, locally within each population analyzed (e.g., in populations representative of African, East Asian, European, South Asian, and Hispanic descent), in order to be considered a qualifying variant.

Additional qualifying variant selection criteria and processing are described in U.S. 2023/0245714, which is herein incorporated by reference in its entirety.

At block 340, the method can include mapping locations of the qualifying variants along a sequence of the gene to variant spatial positions on the three-dimensional protein structure. The regions (e.g., codons) along the gene sequence occupied by each qualifying variant can be determined using the population genomics data for each patient. These regions can be mapped to corresponding spatial positions, such as using the mapping processes discussed above.

In some cases, mapping of the locations of the variants in the sequence of the gene to variant spatial positions on the three-dimensional protein structure can include determining a change in Gibbs free energy of folding from a protein having amino acids coded by the reference genome to a protein having amino acids coded by the variant. In such an example, a change in the absolute value of Gibbs free energy would be determined for each qualifying variant as part of the analysis process. This could be accomplished by a software or modeling tool. In this case, if the sum of differences (or the average difference) of the qualifying variants exceeds a threshold number, then the qualifying variants can be flagged for further analysis.

At block 350, the method can include calculating three-dimensional distances between the candidate spatial position and each of the variant spatial positions on the three-dimensional protein structure.

The distance between the amino acids coded by each qualifying variant and the candidate spatial position can be determined. Specifically, in a first example, calculating the three-dimensional distance includes identifying a three-dimensional position of an amino acid defined by a codon of the qualifying variant, and setting the variant spatial position equal to the three-dimensional position known for the amino acid. The distance is then calculated as a Euclidean distance between the variant spatial position and the candidate spatial position.

In a second example, a qualifying variant includes multiple codons defining multiple amino acids. In such a circumstance, the variant spatial position may be defined as a center point (or centroid) of a point cloud comprising the three-dimensional positions of those amino acids. The center point may be calculated as an average of X, Y, and Z coordinates of each point in the point cloud. Alternatively, the center point may be identified as the median value across all points in the point cloud in X, Y, and Z, or via any other suitable technique for determining a center point of the point cloud. The variant spatial position is set to the center point, and the distance is then calculated as a Euclidean distance between the variant spatial position and the candidate spatial position.

In a third example, a qualifying variant includes multiple codons defining multiple amino acids, and the variant spatial position is defined as the position of whichever amino acid is nearest to the candidate spatial position. The distance is then calculated as a Euclidean distance between the variant spatial position and the candidate spatial position.

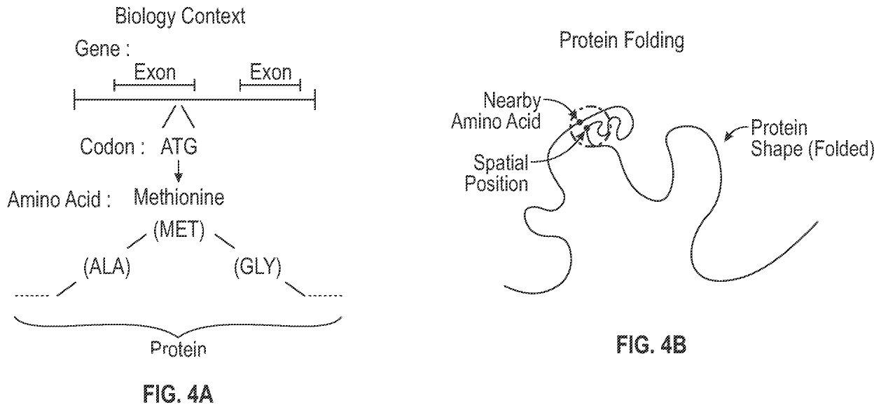

At block 360, the method can include selecting a subset of the variants for inclusion within a statistical window, based on the three-dimensional distances for corresponding variant spatial positions. FIG. 5 illustrates a schematic diagram of such a statistical window selection in an example. Here, regions R1, R2, and R3 are depicted on the gene. Corresponding codons for those regions are depicted on the folded protein.

In a further embodiment, instead of grouping genetic variants together into windows before the analysis, genetic variants can also be analyzed one at a time with regard to the phenotype/trait being considered, and the resulting summary statistics for corresponding variants are combined into the defined windows for analysis to identify which regions of the gene show an association with the trait.

Qualifying variants can be added to the statistical window, starting with those closest to the candidate spatial position in three-dimensional space. The qualifying variants can be added to the statistical window proceeding from closest to furthest away from the candidate spatial position until a predetermined number of individuals carrying the qualifying variant(s) has been reached. For example, until between ten and thirty, or until twenty qualifying variants are added.

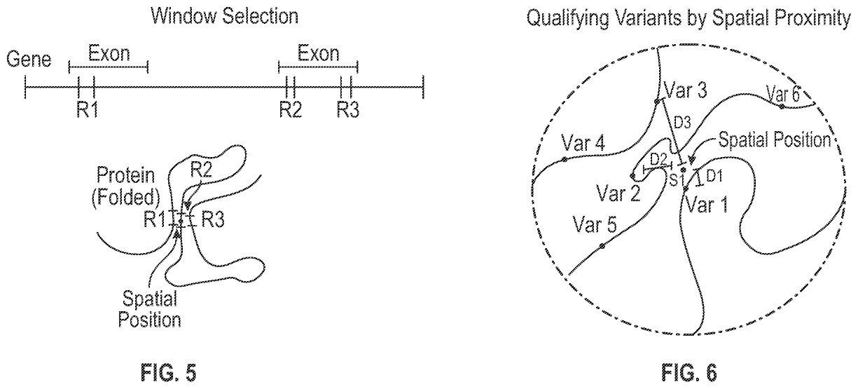

Overall, the variants can be selected and added to the statistical window in order of ascending corresponding three-dimensional distance to the candidate spatial position. A three-dimensional volume of the statistical window can be adjusted to include the variant spatial positions of the subset. FIG. 6 illustrates a schematic diagram of qualifying variants by spatial proximity in an example. Here, distances D1, D2, and D3 have been determined between codons defined by qualifying variants (“var”) 1, 2, and 3, and the candidate spatial position.

The analysis process for the window can proceed, and a next candidate spatial position can be selected. In an example, spatial position of each amino acid at the protein can be considered. In some cases, only a subset of amino acids are considered.

Once the window is set with the predetermined number of qualifying variants, statistical analysis can be done, such as analyzing impact on expression of a phenotype for the selected subset of variants within the statistical window. Example analysis processes for such as statistical window are described in U.S. 2023/0245714, which is herein incorporated by reference in its entirety.

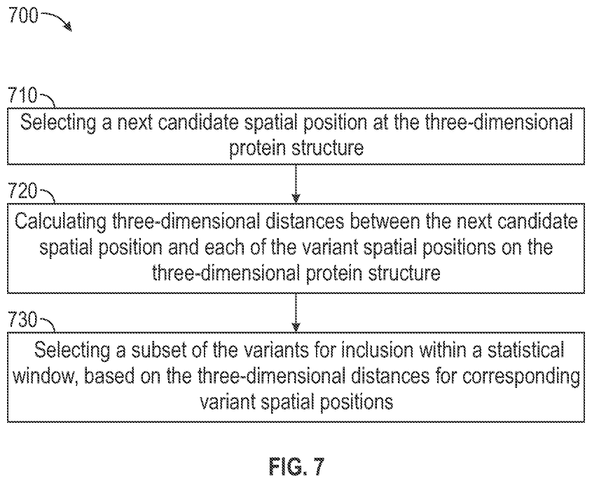

FIG. 7 illustrates a flow chart of a method 700 of iteratively facilitating analysis of variants via consideration of three-dimensional spatial positions on a protein coded by a gene in an example. Here, a modified version of the method 300 can be done in an iterative fashion.

For example, blocks 320 and 350-360 may be performed for each of multiple candidate spatial positions that each comprise a location of an amino acids coded for by a variant in the gene, such that each candidate spatial position corresponds with a different variant and all variants are represented by a candidate spatial position. The processes of distance calculation, qualifying variant selection, and analysis may then iterate across the candidate spatial positions until the position corresponding with each variant has been considered. In this manner, thousands of windows may be constructed (or a window may slide across thousands of positions) to facilitate the analysis of each gene.

First, at block 710, a next candidate spatial position can be chosen at the three-dimensional protein structure. A next candidate spatial position may comprise a spatial position of a next amino acid in the protein, a spatial position of an amino acid coded for by a codon defined by a next variant, a spatial position recited in a predefined list, etc. Then, at block 720, a corresponding three-dimensional distance between the next candidate spatial position and each of the variant spatial positions on the three-dimensional protein structure can be calculated.

Finally, at block 730, a subset of the variants can be selected for inclusion within a next statistical window. These selections can be based on the three-dimensional distances for corresponding variant spatial positions.

### Computer Example

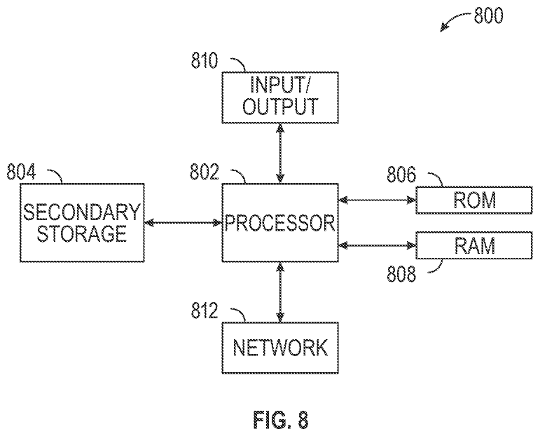

FIG. 8 is a block diagram of a typical, general-purpose computer 800 that may be programmed into a special purpose computer suitable for implementing one or more examples of the manifest record generating program disclosed herein. The manifest record generating program described above may be implemented on any general-purpose processing component, such as a computer with sufficient processing power, memory resources, and communications throughput capability to handle the necessary workload placed upon it. The computer 800 includes a processor 802 (which may be referred to as a central processor unit or CPU) that is in communication with memory devices including secondary storage 804, read only memory (ROM) 806, random access memory (RAM) 808, input/output (I/O) devices 810, and network connectivity devices 812. The processor 802 may be implemented as one or more CPU chips or may be part of one or more application specific integrated circuits (ASICs).

The secondary storage 804 is typically comprised of one or more disk drives or tape drives and is used for non-volatile storage of data and as an over-flow data storage device if RAM 808 is not large enough to hold all working data. Secondary storage 804 may be used to store programs that are loaded into RAM 808 when such programs are selected for execution. The ROM 806 is used to store instructions and perhaps data that are read during program execution. ROM 806 is a non-volatile memory device that typically has a small memory capacity relative to the larger memory capacity of secondary storage 804. The RAM 808 is used to store volatile data and perhaps to store instructions. Access to both ROM 806 and RAM 808 is typically faster than to secondary storage 804.

The devices described herein may be configured to include computer-readable non-transitory media storing computer readable instructions and one or more processors coupled to the memory, and when executing the computer readable instructions configure the computer 800 to perform method steps and operations described above. The computer-readable non-transitory media includes all types of computer readable media, including magnetic storage media, optical storage media, flash media and solid-state storage media.

It should be further understood that software including one or more computer-executable instructions that facilitate processing and operations as described above with reference to any one or all of steps of the disclosure may be installed in and sold with one or more servers and/or one or more routers and/or one or more devices within consumer and/or producer domains consistent with the disclosure. Alternatively, the software may be obtained and loaded into one or more servers and/or one or more routers and/or one or more devices within consumer and/or producer domains consistent with the disclosure, including obtaining the software through physical medium or distribution system, including, for example, from a server owned by the software creator or from a server not owned but used by the software creator. The software may be stored on a server for distribution over the Internet, for example.

Also, it will be understood by one skilled in the art that this disclosure is not limited in its application to the details of construction and the arrangement of components set forth in the description or illustrated in the drawings. The examples herein are capable of other examples, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated examples may be implemented, at least in part, in digital electronic circuitry, analog electronic circuitry, or in computer hardware, firmware, software, or in combinations of them. These components may be implemented, for example, as a computing program product such as a computing program, program code or computer instructions tangibly embodied in an information carrier, or in a machine-readable storage device, for execution by, or to control the operation of, data processing apparatus such as a programmable processor, a computer, or multiple computers.

A computing program may be written in any form of programming language, including compiled or interpreted languages, and it may be deployed in any form, including as a stand-alone program or as a module, component, subroutine, or other unit suitable for use in a computing environment. A computing program may be deployed to be executed on one computer or on multiple computers at one site or distributed across multiple sites and interconnected by a communication network. Also, functional programs, codes, and code segments for accomplishing the techniques described herein may be easily construed as within the scope of the present disclosure by programmers skilled in the art. Method steps associated with the illustrative examples may be performed by one or more programmable processors executing a computing program, code or instructions to perform functions (e.g., by operating on input data and/or generating an output). Method steps may also be performed by, and apparatus may be implemented as, special purpose logic circuitry, e.g., an FPGA (field programmable gate array) or an ASIC (application-specific integrated circuit), for example.

The various illustrative logical blocks, modules, and circuits described in connection with the examples disclosed herein may be implemented or performed with a general-purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computing program include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computing program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory may be supplemented by or incorporated in special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill in the art further appreciate that the various illustrative logical blocks, modules, circuits, and algorithm steps described in connection with the examples disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the disclosure. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor may read information from, and write information to, the storage medium. In the alternative, the storage medium may be integral to the processor. In other words, the processor and the storage medium may reside in an integrated circuit or be implemented as discrete components.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” should be taken to include a single medium or multiple media (e.g., a centralized or distributed database, or associated caches and servers) able to store processor instructions. The term “machine-readable medium” shall also be taken to include any medium, or combination of multiple media, that is capable of storing instructions for execution by one or more processors, such that the instructions, when executed by one or more processors cause the one or more processors to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” as used herein excludes signals per se.

### Various Notes & Examples

In some aspects, the techniques described herein relate to a method for facilitating analysis of variants via consideration of three-dimensional spatial positions on a protein coded by a gene, the method including: receiving a three-dimensional protein structure of the protein encoded by the gene; selecting a candidate spatial position at the three-dimensional protein structure; identifying variants within the gene; mapping locations of the variants along a sequence of the gene to variant spatial positions on the three-dimensional protein structure; calculating three-dimensional distances between the candidate spatial position and each of the variant spatial positions on the three-dimensional protein structure; and selecting a subset of the variants for inclusion within a statistical window, based on the three-dimensional distances for corresponding variant spatial positions.

In some aspects, the techniques described herein relate to a method, wherein the subset of variants is fixed at a number between ten and thirty.

In some aspects, the techniques described herein relate to a method, wherein the variants are selected for the statistical window in order of ascending corresponding three-dimensional distances.

In some aspects, the techniques described herein relate to a method, further including adjusting a three-dimensional volume of the statistical window to include the variant spatial positions of the subset.

In some aspects, the techniques described herein relate to a method, further including analyzing impact on expression of a phenotype for the selected subset of variants within the statistical window.

In some aspects, the techniques described herein relate to a method, further including iteratively: selecting a next candidate spatial position at the three-dimensional protein structure; calculating a corresponding three-dimensional distance between the next candidate spatial position and each of the variant spatial positions on the three-dimensional protein structure; and selecting a subset of the variants for inclusion within a next statistical window, based on the three-dimensional distances for corresponding variant spatial positions.

In some aspects, the techniques described herein relate to a method further including: receiving a reference sequence of the gene; and mapping a plurality of reference locations from the reference sequence to a plurality of spatial positions on the three-dimensional structure protein structure, each reference location among the plurality of reference locations being mapped to a different corresponding spatial position among the plurality of spatial positions; wherein candidate spatial positions are selected from the corresponding spatial positions of the reference locations.

In some aspects, the techniques described herein relate to a method, wherein the calculating of the three-dimensional distance between the candidate spatial position and at least one of the variant spatial positions includes determining the distance between amino acids associated with each codon defined by at least one of the variants and the candidate spatial position.

In some aspects, the techniques described herein relate to a method, wherein the mapping of the plurality of candidate locations from the reference sequence to the plurality of spatial positions on the three-dimensional structure protein structure includes: unfolding the three-dimensional protein structure; and correlating the sequence of the gene to the three-dimensional protein structure; refolding the protein; and determining three-dimensional coordinates of the candidate spatial position.

In some aspects, the techniques described herein relate to a method, wherein receiving the three-dimensional protein structure includes receiving a partially defined three- dimensional protein structure.

In some aspects, the techniques described herein relate to a method, further including determining three-dimensional coordinates defining a location of an amino acid correlating to a codon on a loci of the gene.

In some aspects, the techniques described herein relate to a method, wherein the selecting of the candidate spatial position includes selecting spatial positions identified by a functional screen.

In some aspects, the techniques described herein relate to a method, wherein the selecting of the candidate spatial position includes selecting all spatial positions corresponding with amino acids on the protein structure.

In some aspects, the techniques described herein relate to a method, wherein the selecting of the candidate spatial position includes selecting one or more amino acids corresponding to the candidate spatial position.

In some aspects, the techniques described herein relate to a method, wherein the selecting of the candidate spatial position includes selecting at least one of a gate or a chamber surrounded by amino acids.

In some aspects, the techniques described herein relate to a method, wherein the selecting of the candidate spatial position includes selecting a candidate spatial position located with regard to an external molecule that interacts with the protein.

In some aspects, the techniques described herein relate to a method, wherein the selecting of the candidate spatial position includes selecting one or more interaction sites between regions of two or more proteins.

In some aspects, the techniques described herein relate to a method, wherein the identifying of the variants includes receiving population genomics data indicating locations of genetic variants.

In some aspects, the techniques described herein relate to a method, wherein the identifying of the variants includes selecting variants meeting one or more selection criteria.

In some aspects, the techniques described herein relate to a method, wherein the mapping of the locations of the variants in the sequence of the gene to variant spatial positions on the three-dimensional protein structure includes determining a change in Gibbs free energy of folding from a protein having amino acids coded by the reference genome to a protein having amino acids coded by the variant.

In some aspects, the techniques described herein relate to a method for facilitating analysis of variants via consideration of three-dimensional spatial positions on a protein coded by a gene, the method including: receiving a three-dimensional protein structure of the protein encoded by the gene; selecting a candidate spatial position at the three-dimensional protein structure; identifying qualifying variants within the gene; mapping locations of the qualifying variants along a sequence of the gene to variant spatial positions on the three-dimensional protein structure; calculating three-dimensional distances between the candidate spatial position and each of the variant spatial positions on the three-dimensional protein structure; and selecting a subset of the qualifying variants for inclusion within a statistical window, based on the three-dimensional distances for corresponding variant spatial positions.

In some aspects, the techniques described herein relate to a method, wherein the subset of qualifying variants is fixed at a number between two and one hundred.

In some aspects, the techniques described herein relate to a method, wherein the qualifying variants are selected for the statistical window in order of ascending corresponding three-dimensional distances.

In some aspects, the techniques described herein relate to a method, further including adjusting a three-dimensional volume of the statistical window to include the variant spatial positions of the subset.

In some aspects, the techniques described herein relate to a method, further including analyzing impact on expression of a phenotype for the selected subset of qualifying variants within the statistical window.

In some aspects, the techniques described herein relate to a method, further including iteratively: selecting a next candidate spatial position at the three-dimensional protein structure; calculating a corresponding three-dimensional distance between the next candidate spatial position and each of the variant spatial positions on the three-dimensional protein structure; and selecting a subset of the qualifying variants for inclusion within a next statistical window, based on the three-dimensional distances for corresponding variant spatial positions.

In some aspects, the techniques described herein relate to a method further including: receiving a reference sequence of the gene; and mapping a plurality of reference locations from the reference sequence to a plurality of spatial positions on the three-dimensional structure protein structure, each reference location among the plurality of reference locations being mapped to a different corresponding spatial position among the plurality of spatial positions; wherein candidate spatial positions are selected from the corresponding spatial positions of the reference locations.

In some aspects, the techniques described herein relate to a method, wherein the calculating of the three-dimensional distance between the candidate spatial position and at least one of the variant spatial positions includes determining the distance between amino acids associated with each codon defined by at least one of the qualifying variants and the candidate spatial position.

In some aspects, the techniques described herein relate to a method, wherein the mapping of the plurality of candidate locations from the reference sequence to the plurality of spatial positions on the three-dimensional protein structure includes: unfolding the three-dimensional protein structure; and correlating the sequence of the gene to the three-dimensional protein structure; refolding the protein; and determining three-dimensional coordinates of the candidate spatial position.

In some aspects, the techniques described herein relate to a method, wherein receiving the three-dimensional protein structure includes receiving a partially defined three-dimensional protein structure.

In some aspects, the techniques described herein relate to a method, further including determining three-dimensional coordinates defining a location of an amino acid correlating to a codon on loci of the gene.

In some aspects, the techniques described herein relate to a method, wherein the selecting of the candidate spatial position includes selecting spatial positions that have been identified by a functional screen.

In some aspects, the techniques described herein relate to a method, wherein the selecting of the candidate spatial position includes selecting all spatial positions corresponding with amino acids on the protein structure.

In some aspects, the techniques described herein relate to a method, wherein the selecting of the candidate spatial position includes selecting one or more amino acids corresponding to the candidate spatial position.

In some aspects, the techniques described herein relate to a method, wherein the selecting of the candidate spatial position includes selecting at least one of a gate or a chamber surrounded by amino acids.

In some aspects, the techniques described herein relate to a method, wherein the selecting of the candidate spatial position includes selecting a candidate spatial position located with regard to an external molecule that interacts with the protein.

In some aspects, the techniques described herein relate to a method, wherein the selecting of the candidate spatial position includes selecting one or more interaction sites between regions of two or more proteins.

In some aspects, the techniques described herein relate to a method, wherein the identifying of the qualifying variants includes receiving population genomics data indicating locations of genetic variants.

In some aspects, the techniques described herein relate to a method, wherein the identifying of the qualifying variants includes selecting variants meeting one or more selection criteria.

In some aspects, the techniques described herein relate to a method, wherein the mapping of the locations of the qualifying variants in the sequence of the gene to variant spatial positions on the three-dimensional protein structure includes determining a change in Gibbs free energy of folding from a protein having amino acids coded by a reference genome to a protein having amino acids coded by the variant.

In some aspects, the techniques described herein relate to a non-transitory computer readable medium containing program instructions for causing a computer to perform the method of: receive a three-dimensional protein structure of the protein encoded by the gene; select a candidate spatial position at the three-dimensional protein structure; identify qualifying variants within the gene; map locations of the qualifying variants along a sequence of the gene to variant spatial positions on the three-dimensional protein structure; calculate three-dimensional distances between the candidate spatial position and each of the variant spatial positions on the three-dimensional protein structure; and select a subset of the variants for inclusion within a statistical window, based on the three-dimensional distances for corresponding variant spatial positions.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method for facilitating analysis of variants via consideration of three-dimensional spatial positions on a protein coded by a gene, the method comprising:
receiving a three-dimensional protein structure of the protein encoded by the gene;
selecting a candidate spatial position at the three-dimensional protein structure;
identifying qualifying variants within the gene;
mapping locations of the qualifying variants along a sequence of the gene to variant spatial positions on the three-dimensional protein structure;
calculating three-dimensional distances between the candidate spatial position and each of the variant spatial positions on the three-dimensional protein structure; and
selecting a subset of the qualifying variants for inclusion within a statistical window, based on the three-dimensional distances for corresponding variant spatial positions.

2. The method of claim 1, wherein the subset of qualifying variants is fixed at a number between two and one hundred.

3. The method of claim 1, wherein the qualifying variants are selected for the statistical window in order of ascending corresponding three-dimensional distances.

4. The method of claim 1, further comprising adjusting a three-dimensional volume of the statistical window to include the variant spatial positions of the subset.

5. The method of claim 1, further comprising analyzing impact on an expression of a phenotype for the selected subset of qualifying variants within the statistical window.

6. The method of claim 1, further comprising iteratively:
selecting a next candidate spatial position at the three-dimensional protein structure;
calculating a corresponding three-dimensional distance between the next candidate spatial position and each of the variant spatial positions on the three-dimensional protein structure; and
selecting a subset of the qualifying variants for inclusion within a next statistical window, based on the three-dimensional distances for corresponding variant spatial positions.

7. The method of claim 6 further comprising:
receiving a reference sequence of the gene; and
mapping a plurality of reference locations from the reference sequence to a plurality of spatial positions on the three-dimensional structure protein structure, each reference location among the plurality of reference locations being mapped to a different corresponding spatial position among the plurality of spatial positions;
wherein candidate spatial positions are selected from the corresponding spatial positions of the reference locations.

8. The method of claim 1, wherein the calculating of the three-dimensional distance between the candidate spatial position and at least one of the variant spatial positions comprises determining the distance between amino acids associated with each codon defined by at least one of the qualifying variants and the candidate spatial position.

9. The method of claim 7, wherein the mapping of the plurality of candidate locations from the reference sequence to the plurality of spatial positions on the three-dimensional protein structure comprises:
unfolding the three-dimensional protein structure;
correlating the sequence of the gene to the three-dimensional protein structure;
refolding the protein; and
determining three-dimensional coordinates of the candidate spatial position.

10. The method of claim 1, wherein receiving the three-dimensional protein structure comprises receiving a partially defined three-dimensional protein structure.

11. The method of claim 1, further comprising determining a three-dimensional coordinate defining a location of an amino acid correlating to a codon at loci of the gene.

12. The method of claim 1, wherein the selecting of the candidate spatial position comprises selecting spatial positions that have been identified by a functional screen.

13. The method of claim 1, wherein the selecting of the candidate spatial position comprises selecting all spatial positions corresponding with amino acids on the protein structure.

14. The method of claim 1, wherein the selecting of the candidate spatial position comprises selecting one or more amino acids corresponding to the candidate spatial position.

15. The method of claim 1, wherein the selecting of the candidate spatial position comprises selecting at least one of a gate or a chamber surrounded by amino acids.

16. The method of claim 1, wherein the selecting of the candidate spatial position comprises selecting a candidate spatial position located with regard to an external molecule that interacts with the protein.

17. The method of claim 1, wherein the selecting of the candidate spatial position comprises selecting one or more interaction sites between regions of two or more proteins.

18. The method of claim 1, wherein the identifying of the qualifying variants comprises receiving population genomics data indicating locations of genetic variants.

19. The method of claim 1, wherein the mapping of the locations of the qualifying variants in the sequence of the gene to variant spatial positions on the three-dimensional protein structure comprises determining a change in Gibbs free energy of folding from a protein having amino acids coded by a reference genome to a protein having amino acids coded by the variant.

20. A non-transitory computer readable medium containing program instructions for causing a computer to perform a method of:
receive a three-dimensional protein structure of the protein encoded by a gene;
select a candidate spatial position at the three-dimensional protein structure;
identify qualifying variants within the gene;
map locations of the qualifying variants along a sequence of the gene to variant spatial positions on the three-dimensional protein structure;
calculate three-dimensional distances between the candidate spatial position and each of the variant spatial positions on the three-dimensional protein structure; and
select a subset of the variants for inclusion within a statistical window, based on the three-dimensional distances for corresponding variant spatial positions.
